(19) United States
(12) Patent Application Publication
(10) Pub. No.: US 2007/0197881 A1
(43) Pub. Date: Aug. 23, 2007

(54) Wireless Health Monitor Device and System with Cognition

(76) Inventors: James L. Wolf, Conifer, CO (US); Thomas P. Walker, Morrison, CO (US); Franz Huber, Denver, CO (US); Robert N. Caruso, Evergreen, CO (US)

Correspondence Address: KYLE W. ROST, 5490 AUTUMN CT., GREENWOOD VILLAGE, CO, 80111, US

(21) Appl. No.: 11/678,052

(22) Filed: Feb. 22, 2007

Related U.S. Application Data
(60) Provisional application No. 60/766,963, filed on Feb. 22, 2006.

(51) Int. Cl. A61B 5/00; G06Q 10/00

(52) U.S. Cl. 600/300; 128/920; 705/2

(57) ABSTRACT

A home-based remote care solution provides sensors including a basic health monitor (BHM) that is a measurement and feedback system. The BHM operates with low power integrated communications combined with an in-home, low power mesh network or programmable digital assistant (PDA) with cell phone technology. A cognitive system allows remote monitoring of the location and the basic health of an individual. The BHM measures oxygen saturation (SaO2), temperature of the ear canal, and motion, including detection of a fall and location within a facility. Optionally, the BHM measures CO2, respiration, EKG, EEG, and blood glucose. No intervention is required to determine the status of the individual and to convey this information to care providers. The cognitive system provides feedback and assistance to the individual while learning standard behavior patterns. An integrated audio speaker and microphone enable the BHM to deliver audio alerts, current measurements, and voice prompts. A remote care provider can deliver reminders via the BHM. The device may be worn overnight to allow monitoring and intervention. Through the ability to inquire, the cognitive system is able to qualify events such as loss of unconsciousness or falls. Simple voice commands activate the device to report its measurements and to give alerts to care providers. Alerts from care providers can be in a familiar voice to assist with compliance to medication regimens and disease management instructions. Simple switches allow volume control and manual activation. The device communicates with a series of low-power gateways to an in-home cognitive server and point-of-care (POC) appliance (computer). Alone the BHM provides basic feedback and monitoring with limited cognitive capabilities such as low oxygen or fall detection. While connected to the cognitive server, full cognitive capabilities are attained. Full alerting capability requires the cognitive server to be connected through an Internet gateway to the remote care provider.

### CROSS-REFERENCE TO RELATED APPLICATION

This application claims the benefit of U.S. Provisional Patent Application Ser. No. 60/766,963, filed Feb. 22, 2006, copending.

### BACKGROUND OF THE INVENTION

2. Field of the Invention

The invention generally relates to surgery as applied to diagnostic testing and to computer assisted medical diagnostics. More specifically, the invention relates to monitoring a plurality of physiological data. An aspect of the invention relates to cardiovascular testing and to testing and detecting diverse body conditions. Another aspect of the invention relates to telemetry, such as telemetry by radio, telephone, or computer network.

2. Description of Prior Art

A large segment of elderly and disabled persons who would otherwise require institutional medical care are able to live independently as long as monitoring of their condition and assistance with their needs are provided. Given a trend toward greater independence and convenience of in-home healthcare, this is becoming increasingly important. Providing remote home-based care for high-risk patients typically cared for in hospitals can drive down costs and risks associated with transportation to and from points of care. This has also been shown to improve healthcare access for disabled persons, connect socially isolated individuals to their care providers, and enhance caregiver effectiveness.

Home-based care as described here is not telemedicine, which has yet to fulfill the promise of remote care and appropriate intervention for disease management. European countries seem to be more advanced with the evaluation of fully integrated systems but they still have not achieved a fully deployable system. As reported by Audrey Kinsella, MA, MS Research Director of Information for Tomorrow “The idea of home telehealthcare needs a serious makeover. Even today, home telemedicine or telehealthcare is associated with high-tech, expensive devices and overall inaccessibility for the average home care nurse. We need to get past these perceptions and misunderstandings.”

The term, “home telehealthcare,” is defined as clinician-driven healthcare and education services that are delivered to the home via telecommunications to patients who have already been diagnosed in a standard medical setting. As used herein, the definition further includes other informal caregivers who are interested in monitoring and maintaining the health and welfare of an interested party. The definition also includes forms of communication other than the telephone.

The term, “remote healthcare,” is defined to include this extended form of telehealthcare or home-based care. Remote healthcare is an urgently needed method of caring for individuals who can experience a higher degree of self-care independence when effective monitoring and control is provided. Much of the elderly population and the disabled population fit this description. Persons undergoing transitional care for a treated condition fit this category as well. All such persons will benefit from remote healthcare.

The traditional approach to caring for such individuals relies upon either relatives or care centers such as rehabilitation facilities and nursing homes. This approach is coming under ever increasing pressure due to the fact that relatives are working, thereby diminishing the time available for personal attention to care giving. Also, living in care centers is very expensive. To the extent that remote healthcare can provide an adequate level of in-home monitoring of basic health status, a more cost-effective alternative will have been created for a notable segment of this population without compromising the quality of their care. In addition, staying at home as long as possible is preferred by patients and is generally better for their welfare and spirit.

Communication technologies, from well-known POTS (plain old telephone system) to the Internet, have been used for many years to monitor, diagnose and treat persons remotely. Transmission of information, such as pictures, measurements of blood pressure etc. for diagnosis and treatment is the goal. Medical literature widely reports efforts to provide medical care, remotely. Wireless technologies are starting to be employed in telemedicine as well. However, Audrey Kinsella has identified the need for specialized high-technology knowledge (e.g., rewiring households for advanced telecommunications capabilities, installing sophisticated health care workstations, and requiring a suite of engineers to wait on the doorstep, ready to assist) as impediments to the adoption of telemedicine.

Current wireless technologies employing standards known as 802.11b,g and Bluetooth, used in a low power set of sensors, have significant problems. While 802.11 is successful in the home environment, it is not feasible for low power sensors due to large power consumption and is subject to coverage lapses which can only be found through use. Bluetooth has very limited range and also uses too much power to maintain a connection. The breakthrough in wireless technology known as the ZigBee standard allows devices to route low data rate information through multiple paths to ensure delivery of messages.

It would be desirable to provide an improved method and apparatus for delivering remote healthcare. An improved system of care giving may be based on high technology, but must be easy to use for people without basic computer and electronic experience. A desirable system might not provide every data point to the care provider, but will forward at least events or combinations of events that represent a problem. The underlying technology may be completely hidden from the patient or user.

Desirably, such a system may be enabled by recent developments in computer and telecommunications technology. Most notably, these are: a) affordable computer systems with touch screens and voice response, b) Internet, wireless communications standards of Bluetooth and ZigBee, c) low power electronics providing for long battery life, d) reliable low power GPS sensors and Zigbee triangulation technology, and e) cognitive, learning software systems.

To achieve the foregoing and other objects and in accordance with the purpose of the present invention, as embodied and broadly described herein, the method and apparatus of this invention may comprise the following.

### BRIEF SUMMARY OF THE INVENTION:

Against the described background, it is therefore a general object of the invention to provide a method and apparatus that are capable of enhancing the quality of life for individuals whose mobility or self-care capabilities have been limited due to age or disease. Such an individual may be referred to as the user or patient. More specifically, an object of the invention is to enable such individuals to live in their own homes while receiving monitoring and care. An in-home care provider to monitor and assist in basic health needs may not be available. Many of these individuals are impaired mentally or are on some form of therapy such as oxygen or medication. In this case they are at risk of failure to comply with prescribed therapy, thereby potentially leading to a traumatic event such as falling, loss of oxygen, or loss of consciousness.

Children and other relatives have increasing concerns for the welfare of parents or other family members with limited self-care capabilities. These concerns are becoming manifested in a desire to directly monitor those family member patients and to more actively participate in giving care. These trends create a demand for a new and innovative solution to caring.

A home-based remote care solution must have the following characteristics: (1) Requires little or no understanding of the operation by the individual of the monitoring devices and system. (2) Monitors key physiological parameters relevant to the disease or disability. These parameters include activity level, falls, and key measurements such as SpO2 and consciousness. (3) Provides a determination of patient location, whether in-facility or in-home. (4) Provides cognitive understanding of situations and treatments, based on input from multiple sensors of physiological parameters coupled with interactive coaching of behavior. Inferences must be made utilizing more than one sensor. (5) Provides natural interactions employing speech and provides simple interactions with a point-of-care (POC) appliance and a wearable monitor. (6) Provides full time monitoring capability, both when the patient is in-home and when traveling. (7) Provides a link to a care provider and emergency services.

The invention employs recent technological advances in low power measurements and plug-and-play wireless communications components to create a miniature measurement and feedback system that also provides location determination. Such a device may be called a basic health monitor (the BHM) or the “remote companion” that can accompany a patient throughout his day. Embodiments of the BHM include an earpiece, a pendant, a wrist-mounted BHM, a clip-on BHM for a belt, or pocket-carried BHM. The BHM has low power integrated communications with an in-home low power mesh network, a programmable digital assistant (PDA) with cell phone technology, and a cognitive system. These components allow location determination and remote monitoring of the basic health of an individual.

In the preferred embodiment the BHM will be worn around the ear in the same manner as a conventional hearing aid or the recently introduced Bluetooth wireless headsets or earpieces. The BHM will be able to measure oxygen saturation (SaO2), temperature of the ear canal, and motion, including detection of a fall. A key feature is that no intervention will be required to determine the status of the individual and to convey this information to care providers. A cognitive system provides feedback and assistance to the individual while learning standard behavior patterns.

With an integrated audio speaker and microphone, the BHM is able to deliver audio alerts, current measurements, voice prompts, and reminders provided by a remote care provider. The device may be worn overnight to allow monitoring and intervention both day and night. Through the ability to inquire, the cognitive system is able to qualify events such as loss of consciousness or a fall. Anticipated improvements will allow other measurements to be made such as CO₂, respiration, EKG, EEG and blood glucose.

Simple voice commands can activate the BHM to report its measurements and to give alerts to care providers. Alerts from care providers can be given in a familiar voice to assist the patient with compliance to medication regimens and disease management instructions. Simple switches will allow volume control and manual activation.

The BHM communicates through a series of low-power gateways to an in-home cognitive server and to a point-of-care appliance (the POC), which can be a computer. Acting alone, the BHM provides basic feedback and monitoring with limited cognitive capabilities, such as detecting low oxygen or a fall. While connected to the cognitive server or POC, the BHM attains full cognitive capabilities. Full alerting capability requires the cognitive server to be connected through an Internet gateway to the remote care provider. Using specialized technology within a wireless transceiver of the BHM, the relative position of the BHM within a home or facility may be determined by signal strength triangulation to the gateways.

A key characteristic is the appropriate distribution of intelligence to the BHM through to the cognitive server. BHMs have limited ability to make decisions but in some cases may make decisions on their own, particularly if they are somehow not in communication with the cognitive server. Some decisions may require more information than is available from a single device in order to make decisions. The BHM contains enough sensors within a single unit that some basic decisions such as fall detection may be made standalone. Learning and trend detection require the full cognitive system to make decisions and feedback new detection parameters.

The cognitive system provides high-level qualitative information and quantitative data to the caregiver. The cognitive system compresses data at the remote, in-home location into certain quantitative and qualitative states of health. Because of possible measurement errors and other uncertainties, the architecture of the cognitive system requires communication of health states and outputs as probability distributions. The cognitive system provides two levels of natural interaction with the patient: first, through a primary BHM by speech output and input; and second, through the POC in the home or care facility, by both touch screen and speech interaction.

The cognitive system also contains sensors for non-health parameters that are necessary to the overall safety of the individual patient. These sensors are modular in nature and can be placed according to individually determined need. The sensors can measure multiple parameters such as ambient temperature, surface temperature (as of a cook top), motion, sounds, and infrared signals. The sensors contain a speaker for delivering audio alerts, an LCD display for displaying measurements, and appropriate buttons for interaction. These sensors communicate through a ZigBee wireless connection.

The sensors may be utilized in stand alone capacity, in a network, or in conjunction with a base module in which a sensor module may be docked. Stand alone, a sensor module may interact in different modes, such as wirelessly interacting with another sensor module or with a network controller of a system. A network controller is a special case of a sensor module docked in a 10Base-T base module.

By docking a sensor module into a 10Base-T base module, the sensor becomes part of a wired network of sensors to be consolidated into a set of remote objects.

The POC has integrated communication capabilities along with the cognitive engine. The POC interacts with the user for scheduling activities, medication, and communications with the care provider through integrated phone, voice messaging, email, music, and graphics such as pictures and videos.

The accompanying drawings, which are incorporated in and form a part of the specification, illustrate preferred embodiments of the present invention, and together with the description, serve to explain the principles of the invention. In the drawings:

### BRIEF DESCRIPTION OF THE DRAWINGS

FIG. 1 is an isometric front right side view of a basic health monitor (BHM), showing representative locations of subcomponents.

FIG. 2 is a view similar to FIG. 1, showing a BHM from the left rear.

FIG. 3 is a schematic view of an overall remote healthcare system, showing a BHM and modular sensors associated with the patient and an in-home setting.

FIG. 4 is a functional block diagram of the internal components of a BHM, sensor, or similar modular device, showing functional interactions.

FIG. 5 is a schematic communications level diagram showing software components and a communications path from a BHM through a gateway to a cognitive server.

FIG. 6 is a schematic block diagram of the cognitive operation software components of the BHM and cognitive system.

FIG. 7 is an isometric view of a modular sensor device, taken from bottom front.

FIG. 8 is an exploded view of the sensor of FIG. 7, showing suggested component locations.

FIG. 9 is a view similar to FIG. 8, taken from top rear.

FIG. 10 is an isometric view taken from front right, showing a sensor attached to a base module.

FIG. 11 is an isometric view of an alternate embodiment of a BHM, taken from the front lower left side, showing a pendant or belt clip mounted BHM.

FIG. 12 is a view similar to FIG. 11, taken from upper right rear of the alternate embodiment of the BHM.

FIG. 13 is a plan view of a remote healthcare system installed in a home, schematically showing the patient and a method of determining location.

FIG. 14 is a front isometric view of the POC, showing interface components.

### DETAILED DESCRIPTION OF THE PREFERRED EMBODIMENT

The invention relates to a remote healthcare delivery system that includes a basic health monitor (hereinafter “BHM”). The delivery system further includes a network of sensor modules that enables home-based care of independently living elderly and disabled persons, who will sometimes be called the “patient” or “user.” The BHM and sensor modules are similar to one another, with the BHM being primarily adapted to be worn by the patient while the sensor modules are primarily adapted to be distributed in the patient's home or care facility.

The invention contemplates that a natural network surrounds a person or patient. Such a network may include both professional caregivers and other support individuals who might provide care on an informal basis. The informal caregivers are relatives, friends, co-workers, and/or neighbors. The professional caregivers are the individual's network of doctors, nurses, emergency medical technicians, etc.

Another portion of the invention for delivering remote healthcare is a cognitive system to evaluate health parameters and trends. Prior telehealthcare systems have not included this ability. A cognitive system can reduce the demands that the delivery of raw data otherwise places upon the informal care givers, thereby avoiding a portion of false alarms. A cognitive system can work together with all sensors within the remote healthcare system, especially with the BHM sensor. The BHM measures basic health function such as pulse rate, temperature, oxygen saturation, movement, acceleration, and location. The BHM also contains a speaker and microphone for speech interaction. The patient wears the BHM at all times. Through the speaker and microphone built into the BHM, the cognitive system is able to give prompts to the patient and can receive answers from the patient. This ability is crucial for implementing the cognitive, learning software included in this remote healthcare system and for enabling the prompting features of the system.

A remote healthcare delivery system must fulfill three needs: a) safety, b) security, and c) social needs. Safety issues to be monitored include basic health assessments such as oxygen saturation, blood pressure, appropriate movement, and so on. Security is defined by the status of doors open/closed, appliances on/off, temperature in the house and so on. The importance of social interaction for the physical as well as emotional well being of the patients is becoming increasingly evident. Appointments for social and recreational activities and integrated communications form the basis of fulfilling these social needs. Information about safety, security, and social needs must be current, accurate and readily available both to the patient and to the person(s) involved in assisting him.

A network of sensors, including the BHM, is located throughout the patient's dwelling. The sensors track and monitor the patient's health status and activities. The sensors provide input for proactive applications that will offer a variety of assistance, ranging from reminders to take medications to accessing social support. The patient will access this network through a point-of-care appliance, hereinafter called “POC,” by using a variety of familiar interfaces, such as integrated calendar, telephone, and simplified email that utilize appropriate assistive technology. The patient will not need to learn new technology to receive assistance. These proactive systems enable relatives to assess the health and well-being of the patient remotely through private, secure Internet connections and will provide social support to on-site caregivers. Such social support to caregivers is necessary to avoid burnout, which is a common problem among caregivers.

The remote healthcare delivery system is distributed, which in certain circumstances might risk a full or partial loss of communications. In order to ensure that the system is robust, the cognitive intelligence also is distributed, especially to the BHM. A fusion of the data from the network sensors enables a feedback of the patient's health state. This fusion enables an adaptive intelligent assistance to the patient even when there is a communication failure.

The remote healthcare delivery system employs a mesh network, which enables a new approach to care for the patient. To date, most wireless systems have employed cellular-phone-type radio links implementing point-to-point or point-to-multipoint transmissions. These prior networks are difficult to install, configure and maintain. Also, they are highly vulnerable to failure, thereby leading to dropped signals. In contrast, wireless mesh networks are multi-hop systems, where the components assist each other in transmitting signals. Signals may take several hops through different components to reach their intended destination. Mesh networks are especially well suited to adverse conditions and are easy to install, self-configuring, and self-learning. Devices can be added to a mesh network without technical knowledge and by following simple installation instructions. This makes them particularly useful for the type of care, specific application, and targeted users as identified herein.

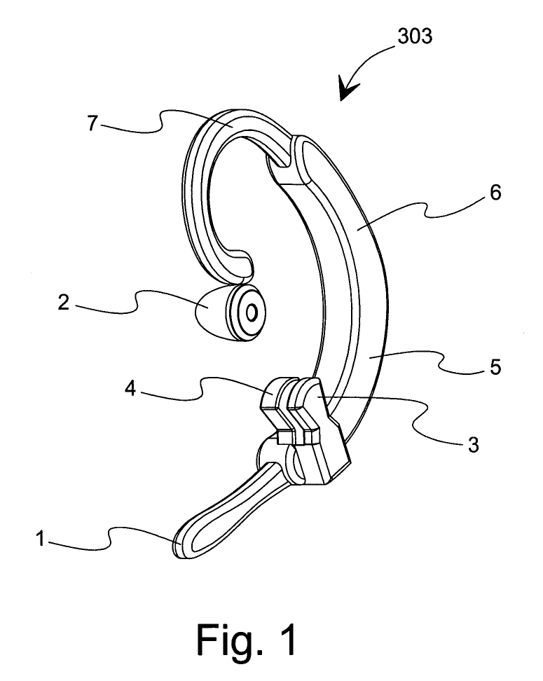

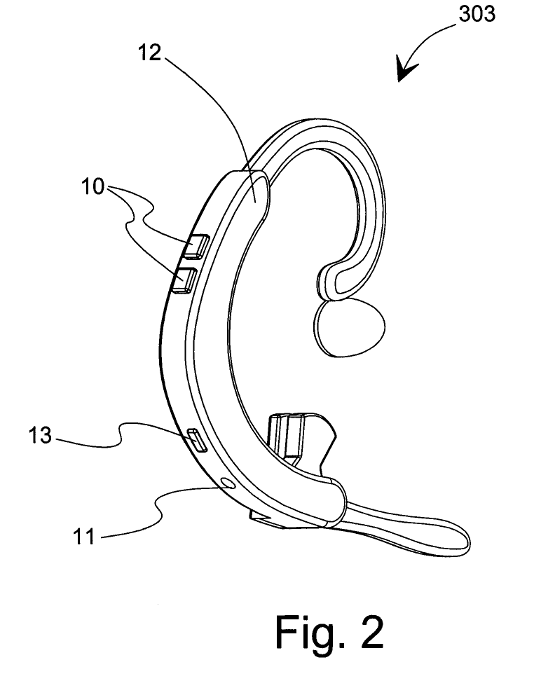

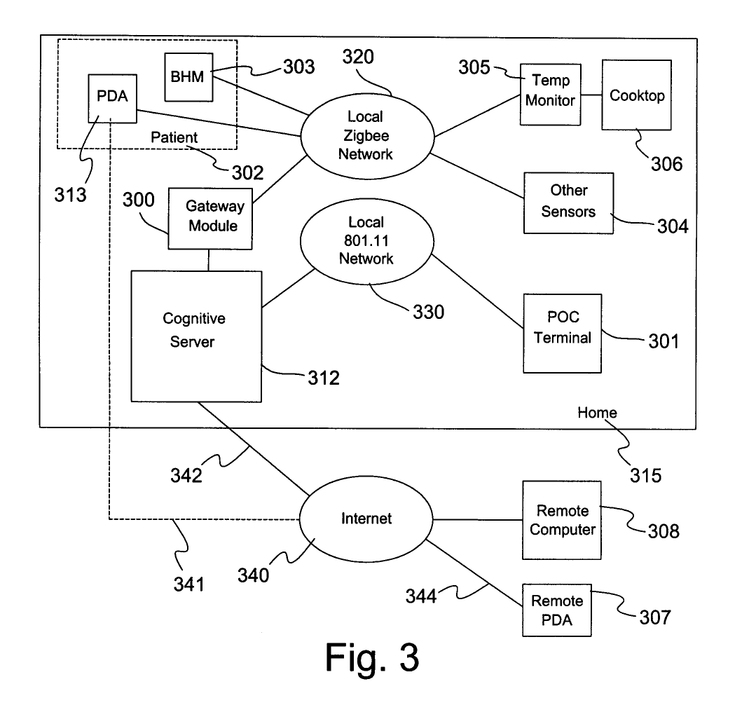

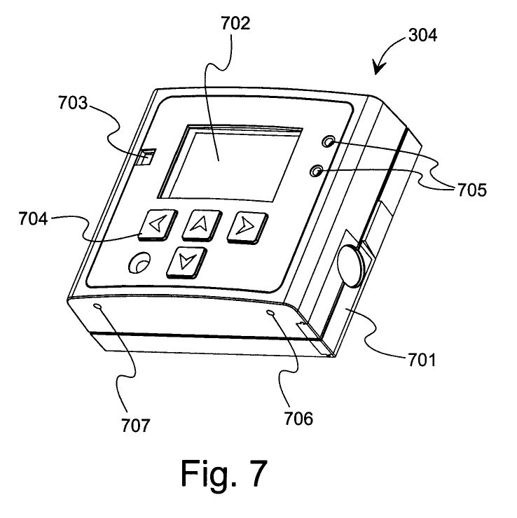

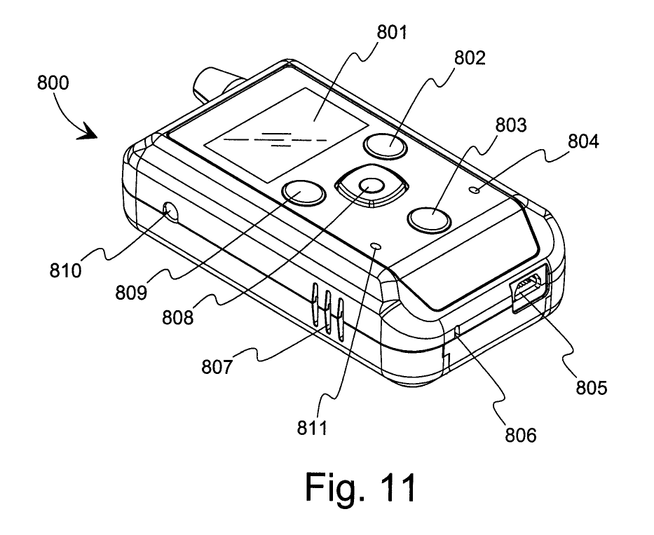

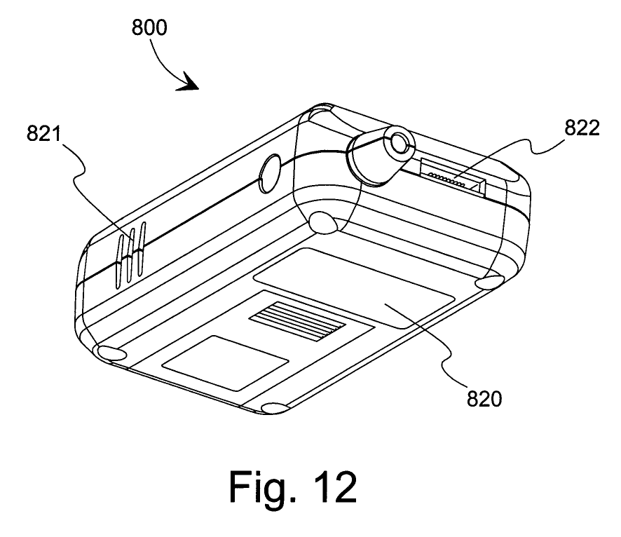

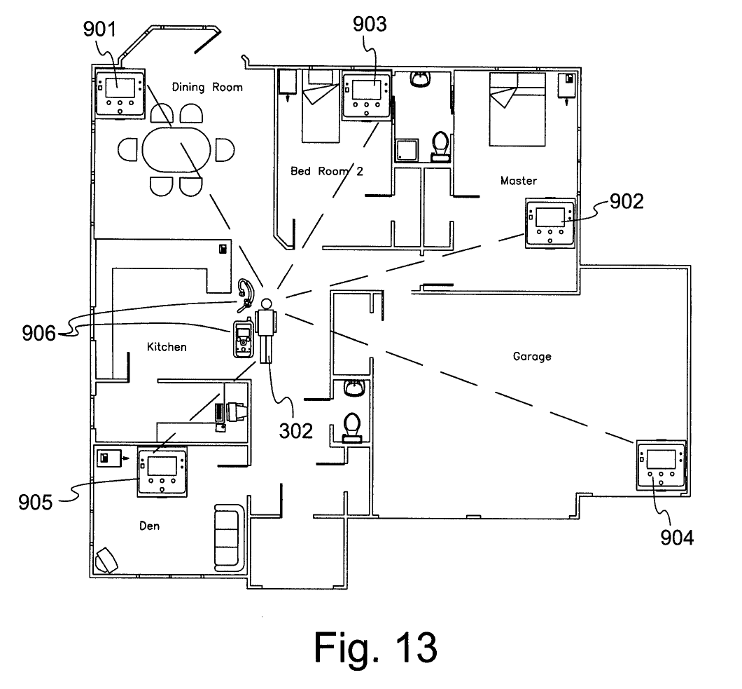

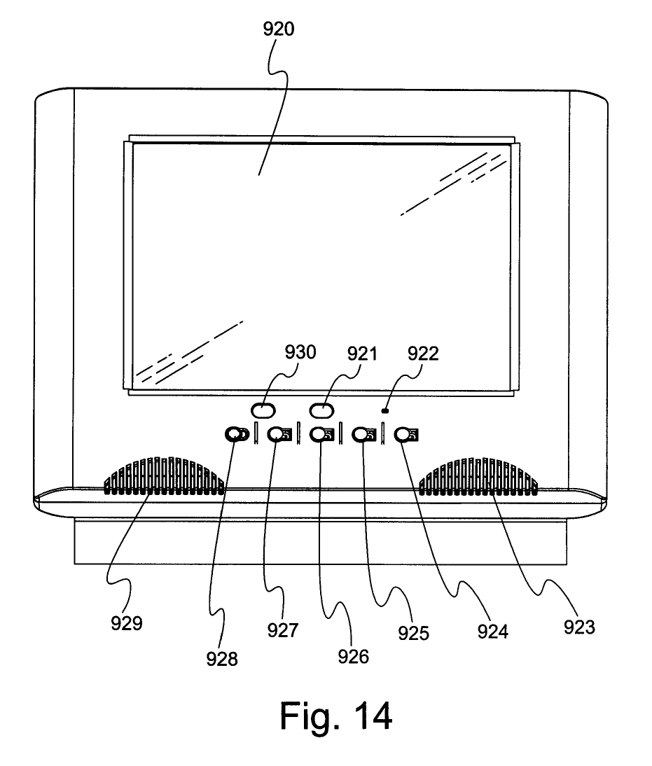

In the following detailed description, one communication path may be described for use by any particular component. Such descriptions should be understood to be representative. Many of the measurement components may follow similar communication paths. Therefore, all disclosed communication paths are applicable to each component and for communicating each measurement. In the following description, the numbers from 1-99 are elements primarily shown in FIGS. 1-2, numbers of the 300, 400, 500, and 600 series refer to elements primarily shown in FIGS. 3, 4, 5, and 6, respectively. Numbers of the 700 series refer to elements primarily shown in FIGS. 7-10. Numbers of the 800 series refer to elements primarily shown in FIGS. 11-12 and numbers in series 900 refer to elements primarily shown in FIGS. 13 and 14.

FIGS. 1 and 2 show a basic health monitor (BHM) 303 of a form factor suited to be wearable. As suggested in these figures, a preferred configuration of the BHM 303 is as an earpiece. A BHM 303 contains subcomponents that enable various functions. Other configurations of the BHM perform similar functions and contain similar subcomponents. In a BHM of the form factor in FIGS. 1 and 2, many of the subcomponents are internal. Thus, various subcomponents are identified as representative locations on the earpiece 303. The subcomponents are microphone 1, earphone and temperature sensor 2, dual light emitting diodes (LEDs) 3, optical sensor 4, accelerometer 5, microprocessor 6, and antenna 7, all as shown in FIG. 1. FIG. 2 shows additional components including volume controls 10, indicator LED 11, ZigBee radio transceiver 12, and on/off button 13. The LEDs 3 and optical sensor 4 are spaced apart, and the configuration of the earpiece 303 is suitable for the user's earlobe to be located between the LED's 3 and optical sensor 4 to enable measurements more fully described below.

The wearable BHM 303 and other system elements in the home are shown schematically in FIG. 3. A boxed portion 315 of the figure represents the home or care center and shows which components are found within the home 315 or care center. Within the home 315, a smaller boxed portion 302 represents the patient and shows devices such as the BHM 303 that the patient 302 carries or wears. Of course, the patient 302 is mobile and may leave the home, taking such devices 303 with him. This figure also shows multiple communication paths represented as ellipses. These are a Zigbee wireless path 320, a wired or wireless 801.11 path 330, and an Internet path 340, which may be by wired line 341 or a wireless cellular network 344. Lines connecting each device in the figure represent a communication path, with lines to an ellipse representing a connection to the respective network.

One or more point-of-care (POC) appliances or computer terminals 301 are located in the patient's home for the patient's use. A POC 301 has full touch screen and voice interactive capabilities and communicates through a local network 330 with a cognitive server 312.

A router gateway module 300 has a USB link to the cognitive server 312. The router gateway module 300 provides a communication bridge from the wireless Zigbee network 320 to the network 330 through the cognitive server 312. This bridge allows communications with the patient 302 via the wearable basic health monitor 303 through a Zigbee connection. Additional wireless Zigbee modular sensors 304 are deployed at other locations in the house. As a specific example, the additional sensors 304 may include a modular surface temperature sensor 305 that is located to monitor a cooking surface or range 306. The router gateway module 300 and sensor modules 304 are similar.

The remote healthcare delivery system includes components operative outside the home 315. When the patient 302 is outside of the home, the accompanying BHM 303 communicates through Zigbee network 320 to the optional programmable digital assistant (PDA) 313, which the patient 302 carries with him. The PDA 313 communicates with the cognitive server, either through the link 341 or through a cellular connection to the Internet, in turn linking by connection 342 to the cognitive server 312. The cognitive server 312 communicates through the Internet 340 to one or more desktop remote computers or patient monitors 308 located at a remote caregiver site. The remote healthcare system may include a remote PDA or remote patient monitor 307 connected through the Internet by cellular network link 344.

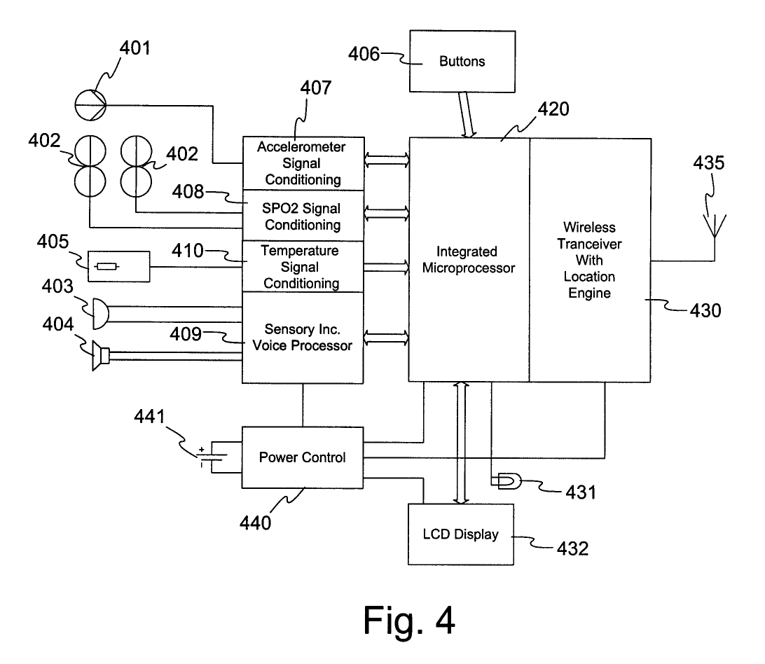

The general interactions and structure of BHM 303 and the similar or parallel portions of sensor module 304 and the like are shown in FIG. 4. A miniature accelerometer sensor 401 communicates with accelerometer signal conditioning circuitry 407. A dual light emitting diode oxygen saturation (SpO2) sensor 402 communicates with a SpO2 signal conditioning circuitry 408. A microphone 403 and speaker 404 communicate with speaker/earphone conditioning circuitry 409. A preferred component to serve as or to substitute for conditioning circuitry 409 is a Sensory Inc. voice processor 409 (Sensory, Inc., 575 N. Pastoria Ave., Sunnyvale, Calif.). Temperature sensor 405 communicates with temperature and signal conditioning circuitry 41 0. Buttons 406, such as volume on/off buttons, control power or functionality to an integrated microprocessor 420. The microprocessor 420 communicates with a wireless ZigBee radio transceiver 430, which operates through an appropriate antenna 435. The transceiver 430 contains a location engine, described below. LED indicators 431 and an LCD display 432 that is optional on some form factors of the BHM provide indications of selected modes and operations. A battery 441 provides power via appropriate power circuitry 440.

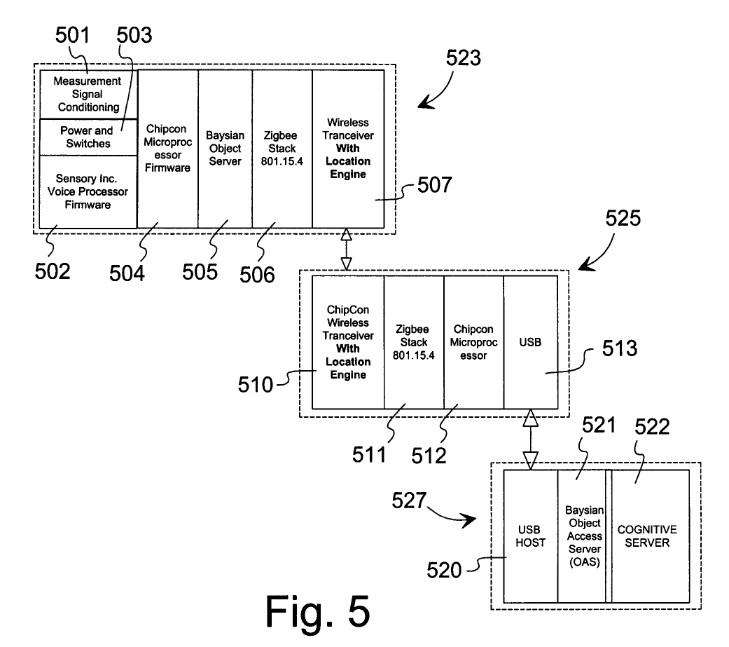

FIG. 5 shows software and communications function of a BHM 523 or similar sensor, through functions of a Zigbee gateway module 525, interacting with functions of a cognitive server 312 through a ZigBee gateway. The BHM 523 includes a microprocessor that executes a main operating program 504 from firmware. The main operating program 504 causes periodic measurements to be taken at preselected times or intervals, without requiring external polling from the cognitive server. The preferred microprocessor is a Chipcon (trademark of Chipcon AS, Gaustadelléen 21, No-0349, Olso, Norway). A measurements software subroutine 501 provides measurement signal conditioning. A power control subroutine 503 controls power and switches.

An audio input and output subroutine 502 executes on a separate Sensory Inc. (575 N. Pastoria Ave., Sunnyvale, Calif.) speech recognition microprocessor and provides audio signal conditioning, speech recognition, and output. The Sensory, Inc. voice processor 502 is linked via a serial digital interface to the Chipcon microprocessor 504.

A Baysian Object server 505 provides standard interfaces to the remote system, which includes the remote patent monitors 307, 308 and POC 301. The Baysian Object server 505 includes a ZigBee and 801.15.4 communications stack 506. A Chipcon (trademark of Chipcon AS, Gaustadelléen 21, No-0349, Olso, Norway) wireless transceiver 507 in the Baysian Object server intercommunicates by an appropriate subroutine with another Chipcon wireless transceiver 510 in a ZigBee gateway 525.

The ZigBee gateway 525 includes a ZigBee and 801.15.4 communications stack 511, a microprocessor 512 operating a main program for the gateway, and a USB interface 513 providing interface to a PDA or desktop computer such as the cognitive server 527. The USB interface 513 preferably provides intercommunication with a desktop computer 527 with an EmWare (trademark of EmWare, Inc., 6322 S. 3000 E, Ste 250, Salt Lake City, Utah 84121) distributed object controller or equivalent, which includes a USB host controller 520 that provides an interface to the subroutine in the desktop or PDA. In addition, the PC server or cognitive server 527 includes a Baysian object access server 521 that carries out Baysian object interface subroutines to the cognitive server 522.

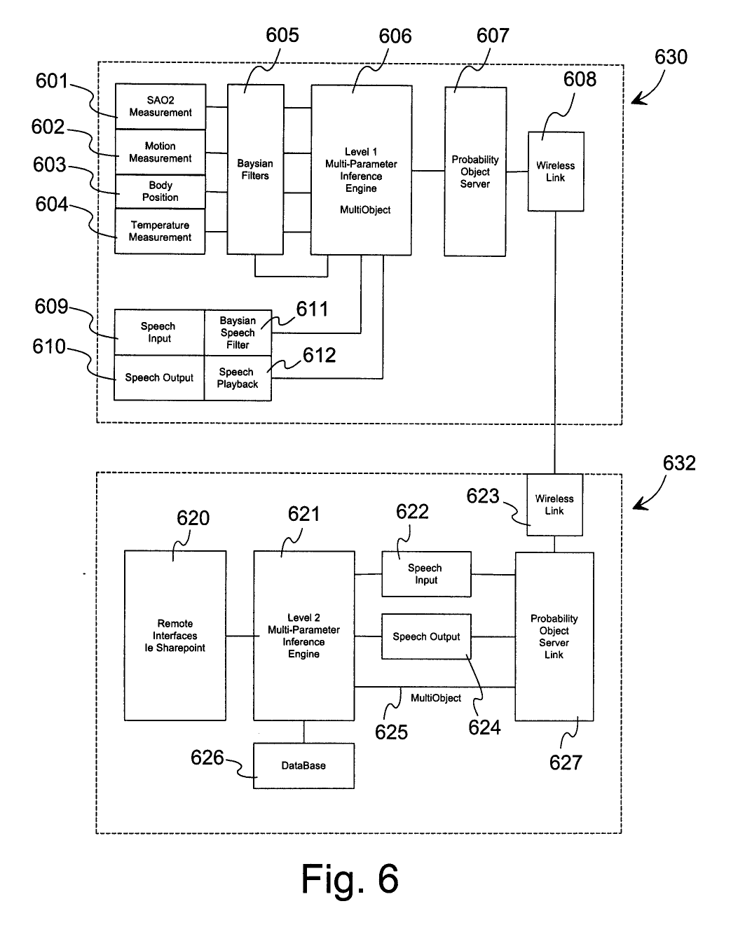

Cognitive operation software components of the various devices and system are shown in FIG. 6. An upper block 630 is the BHM software block diagram. A lower block 632 is the PC software block diagram showing the cognitive server. BHM software routines include SpO2 measurement subroutine 601, a motion measurement subroutine 602, a body position measurement subroutine 603, and a temperature measurement subroutine 604. The subroutines communicate through Baysian filters 605, consisting of statistical filter subroutines, with a level one multi-parameter inference engine 606 within the BHM. A probability object server 607 carries out Baysian probability distributions object server subroutines and communicates through a wireless link 608 carrying out wireless communications subroutines inclusive of the ZigBee software stack 506 and the Chipcon wireless ZigBee transceiver 507.

The ZigBee gateway is a wireless link from BHM or similar sensor module to the server 527, inclusive of elements 510-513, and intercommunicates with the wireless link 608 of the BHM. Included software components are remote interface link subroutines 620 such as Sharepoint (trademark of Microsoft Corporation, One Microsoft Way, Redmond, Wash. 98052) services, a level two multi-parameter inference engine outside device 621, speech input processing subroutines 622, speech output processing subroutines 624, Basian Object interface subroutines 625, a database 626 for storing inference expertise and learning, and a Basian Object server link 627.

FIG. 7 shows details of a modular device 304 used as gateways to the BHM device or as part of the location triangulation feature. Such a device is battery powered and is equipped with a battery compartment within battery cover 701. A liquid crystal diode (LCD) display 702 provides selected readout. A photo sensor 703 provides useful determination of day or night conditions or the state of a lamp or light. Function keys 704 provide input and selection of functions. Indicator LEDs 705 confirm settings and operation. A microphone port 706 and speaker port 707 enable input and output of audible communications.

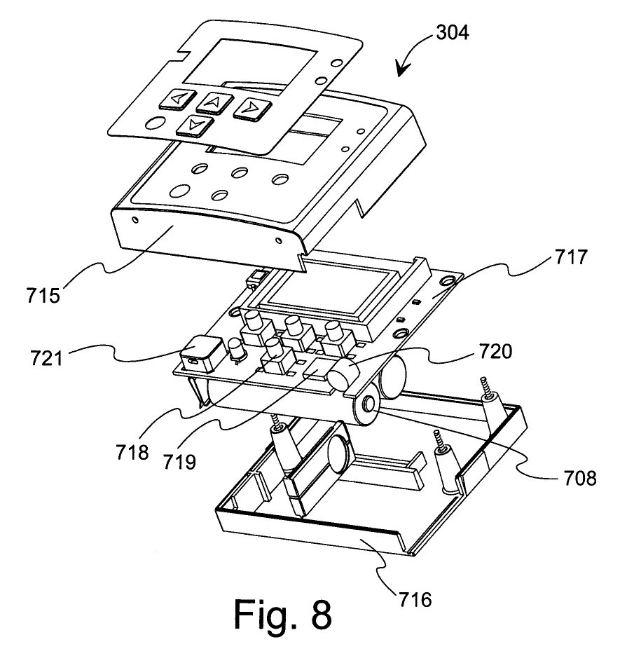

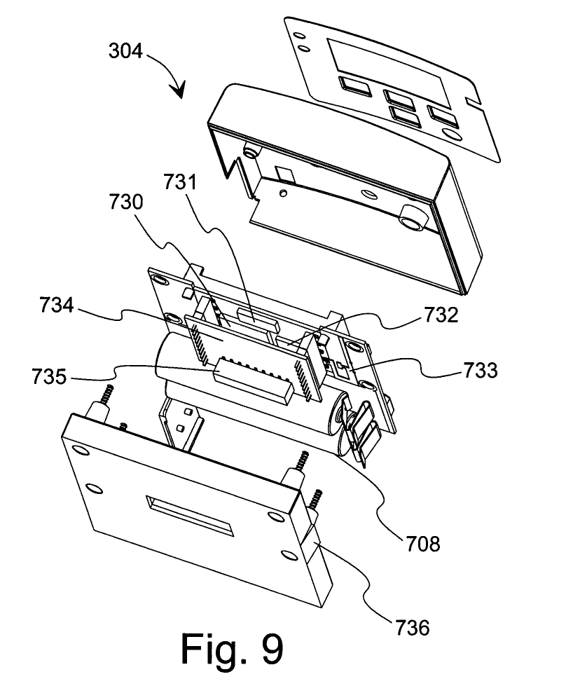

FIGS. 8 and 9 show component locations of a modular device 304. Front cover 715 and back cover 716 contain a main printed circuit board (PCB) 717 that carries input buttons 718, accelerometer 719, microphone 720, and speaker 721. The PCB 717 also carries batteries 722. As best shown in FIG. 9, the PCB carries a microprocessor 730, a radio transceiver 732, an on board antenna 733, and a connector to LCD display 731. The PCB may carry a daughterboard 734 with interface connector 735. Back cover 716 carries a base connector 736.

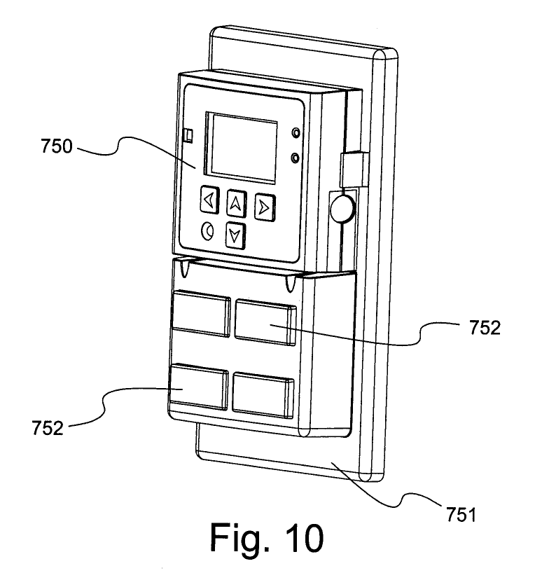

FIG. 10 shows the modular device 750 attached to a wall-mount base 751 having buttons 752 which operate device functions and provide the same functions as buttons 704.

In greater detail, wearable BHM 303 fits on the ear of an individual patient 302. The BHM device measures oxygen saturation SpO2 and cardiac pulse through the dual led 3 and optical sensor 4 across the patient's ear lobe membrane. The BHM is reversible so that it can operate on either ear. The measurements are made periodically under timing control of the microprocessor 6, which has been given instructions from the cognitive server 312. The period of measurement is optimized for low power consumption and necessary physiological needs by cognitive server 312.

The BHM measures inner ear temperature through a sensor within earpiece 2. Also within the earpiece 2 is an earphone for delivering speech and audible alerts from microprocessor 606, FIG. 6, through the speech microprocessor 409, FIG. 4, which incorporates firmware 502, FIG. 5, implementing a speech playback system 612, FIG. 6, through output amplifier 610 through the combined speaker and earphone 404, FIG. 4. Speech input is recognized and communicated through the microphone 1, 403, 806 (FIG. 11) to a speech input amplifier 609 and a Baysian speech filter 611 serving as a speech input decoding system that operates within the speech microprocessor 409 with firmware 502 and through to the cognitive server 312.

The cognitive system is partially contained in the BHM and partially in remote server 312. Sufficient capability to deliver emergency commands to the patient in the event of disconnection from the cognitive server 312 is contained with the BHM utilizing the Sensory, Inc. speech microprocessor 502. The speech input is normally forwarded to the cognitive server 312 for full voice recognition. The level one cognitive server may recognize a few key words through the BHM's Baysian speech filter 605 for requesting simple information such as current SpO2 readings.

A miniature three-axis accelerometer 5 is contained within the BHM to measure the position of the patient's head, motion from normal activities, and motion from extraordinary events such as a fall. Input from this sensor is processed by accelerometer circuitry 407 to the microprocessor 6. Subsequently the input is processed to the cognitive system through a two-way wireless ZigBee communications path that includes the transceiver 12, 430, antenna 7,435, and the gateway router 300 to a mirror image transceiver 623 in the gateway. Firmware in the BHM will quantify the three-axis orientation of the individual and the relative motions of the head. These motions and position will feed into a Baysian filter 605 to determine a first level inference as to activity level and fall detection. This inference may be used on the BHM, if it is not connected to the cognitive server, to deliver emergency alerts to the individual. When connected to the cognitive server through the wireless communication path, the information about position and activity is forwarded to the cognitive server's inference engine 621 for a more complete determination of the importance of the current values of these measurements.

Similarly, the temperature measurement and oxygen saturation (SpO2) of the individual are processed through the system.

The cognitive server 632 has full multi-parameter inference engine 621, meaning some inferences as to the current health of an individual must be made by using multiple measurements and relating them through the Baysian inference filters. For example, the inference engine 621 determines a fall from multiple measurements, which may include the position from which the individual started and ended, and the relative accelerations in between. These and other measurements may be necessary for determining the difference between lying in bed and falling on the floor. Additional measurements such as pulse rate and oxygen level may qualify the fall with a determination of expected consciousness.

A significant event detected through the cognitive system may cause the delivery of an interactive session with the individual to further refine and learn the appropriateness of the determination. For example, a detected fall may deliver a question in the form of voice message to the individual, “Have you fallen?” If no answer is given the determination probably is: The person has fallen and is unconscious. Or if an answer of yes is given, this will validate the Baysian filter coefficients. If the answer is no, then a correction to the Baysian filters will be made and forwarded to the BHM for future fall detection. Thus a key feature of this system is the use voice and other interactions to update its ability to provide correct detection of different health related incidents. Many of these events may evidence themselves over a long period of time, such as SpO2 deteriorating through lack of oxygen availability. One of the functions of the cognitive server inference engine is to evaluate these trends to determine if there is cause for an alert or alarm.

Upon detection of a significant health risk, the system may alert the appropriate monitoring caregiver through the remote POC interfaces 307, 308, and 920 (FIG. 14).

Other sensors may include a modular sensor 304 and, as a specific example, a modular surface temperature sensor 305. Such modular sensors may be used within a monitored environment such as a home to monitor various ambient situations to ensure the safety of the individual. These sensors incorporate the same basic design of electronics, microprocessor, firmware and audio wireless communication as previously described. For example, the temperature sensor 305 may monitor a cook top 306, which is a leading cause of fires within the home of an elderly person who may forget to turn off the cook top. Upon detecting of a rise in cook top temperature, the cognitive system will begin monitoring the appropriateness of the length of time that the cook top is on. Upon a determining an inappropriate behavior, the cognitive system issues an alert to the individual followed by an alert to the caregiver. This alert may be made through a BHM wireless module, another wireless sensor module 304 in the same room, or the POC appliance 301 in the kitchen or other convenient location. The alert may be issued in the form of a voice message, “Did you intend to leave on the cook top.” Once again through a voice interaction through the BHM, a module or the POC appliance or GUI on the POC appliance, an improvement will be made in the Baysian algorithms determining this event.

While the patient 302 is outside of the home environment, a programmable digital assistant (PDA) 313 cellular device with a ZigBee wireless transceiver and global positioning system (GPS) will maintain the BHM device and other body sensors to the cognitive server and inference engine. A portion of the cognitive functioning of the cognitive server will be duplicated in the PDA such that a more immediate response and speech interaction is possible. When the primary link to the cognitive server is not possible, a backup link to POC appliances can be made for alerts.

Various alternative embodiments of the BHM are contemplated. In certain situations the BHM may not be worn long-term on the ear and may alternatively be manufactured in the form of a wristwatch or pendant with a clip for attaching to a belt. FIGS. 11-12 show health monitor 800, which is the size of a small cell phone, and is suitable to be carried or worn as a pendant or clipped to a belt. Similar components are contained within the pendant device 800 as in the ear mounted BHM. Components include microphone 806, speakers 807 and 821, LCD display 801, function buttons 802, 803, 809, function toggle button 808, USB interface connector 805, and external SPO2 sensor connector 822. A port 810 for an external speaker and microphone enable the use of an external, hands free headset, as desired. Ports 804 and 811 allow connection of individual external microphone or speaker, respectively. A mounting 820 for a belt clip is located on the rear face of the health monitor 800.

Internal components not shown are similar to the ear-mounted device. These include a microprocessor, a Zigbee RF transceiver, an antenna, a speech processor, accelerometer, a temperature sensor, a battery, and support electronics. Some measurements will not be able to be made solely by this device but will require other sensors on the patient's body to be wirelessly linked to it.

Other wireless sensors on the body will form a wearable sensor network connected to the cognitive server and BHM. Additional sensors may include, but are not limited to, SPO2, electrocardiogram, blood glucose, and respiration.

FIG. 13 represents a typical home with an individual 302 wearing or carrying a BHM companion 906, illustrated to be a BHM of any form factor. The individual is able to move freely about while maintaining communications through the fixed location gateway modules 901,902,903,904 and 905. These gateway modules may be selected from gateway 300, sensor module 304, or sensor module 305. Each of these modules, including the BHM companion 906, contains the CC2431 Chipcon Zigbee RF Transceiver 430, 507. This Chipcon transceiver contains Chipcon's proprietary circuitry for determining the relative position of BHM 906 to gateway modules 901,902,903,904 and 905 through RF signal strength and triangulation, suggested by the dashed lines between the gateway modules and the position of patient 302. This position is communicated through the gateway modules to the cognitive server.

Modular sensors 304, 305, and the like include those for motion, infrared, light levels, occupancy, ambient temperature, door position including garage door, and medication delivery. The modular devices also have the capability to be wired into control circuits such as garage door openers and thermostats.

FIG. 14 shows in more detail the key components of the integrated communications appliance 301 (POC) in the system diagram of FIG. 3. The POC 301 is a customized Intel based computer optimized for use as an appliance. The LCD screen 920 is utilized to display the pertinent activities for the individual under care. The LCD screen also contains a touch interface to eliminate the need for a mouse. The activities calendar, phone, email, voicemail, and graphics such as pictures are presented for the individual 302 to view on screen 920. Stereo speakers 929 and 923 are utilized for audio alerts, delivery of voice prompts, voice mail, email, music and the integrated speakerphone output. An integrated microphone 922 is likewise utilized for voice input and detection of sounds.

Proximity sensor 921 is utilized to determine the presence of an individual in front of the POC. The detection of an individual's presence is utilized to trigger certain interactive activities such as the notification of email, voicemail and reminders. Buttons 924-928 are utilized as quick entry into different screens such as phone, email and calendar. An integrated camera 930 views activities within its field of view. A key feature of this device is software that is completely integrated and optimized for easy use and cognitive capabilities.

The foregoing is considered as illustrative only of the principles of the invention. Further, since numerous modifications and changes will readily occur to those skilled in the art, it is not desired to limit the invention to the exact construction and operation shown and described, and accordingly all suitable modifications and equivalents may be regarded as falling within the scope of the invention.

What is claimed is:

1. A distributed cognitive system adapted to deliver remote healthcare through medical monitoring and care intervention to a home-based patient, comprising:
a health monitor having a form factor suitable to accompany a patient and adapted to measure a plurality of patient health parameters;
a plurality of sensor modules suitable for deployment about the patient's surroundings and adapted to measure ambient parameters;
a local computer operating software establishing a cognitive system capable of receiving measurements of patient health parameters and measurement of ambient parameters, determining trends therefrom, reaching healthcare decisions, and issuing prompts to the patient; and
means for local bidirectional wireless communication among said health monitor, said sensor modules, and said local computer, enabling exchange of measurement results and feedback of prompts;
wherein, the health monitor operates a distributed portion of the cognitive system such that the health monitor is capable of reaching patient healthcare decisions based on said health parameter measurements and of providing assistance derived from said decisions.

2. The distributed cognitive system according to claim 1, wherein:
said plurality of sensor modules operates a distributed portion of the cognitive system such that the sensor modules are capable of reaching decisions concerning ambient conditions based on said ambient parameter measurements and are capable of interactively sharing information over said bidirectional communication means to locate said health monitor.

3. The distributed cognitive system according to claim 1, wherein said health monitor further comprises:
means for delivering audible alerts, voice prompts and messages from the cognitive system to the patient;
means for recording audible response from the patient;
processing means for interpreting measurements and delivering feedback responses.

4. The distributed cognitive system according to claim 1, wherein:
said health monitor has the form factor of an earpiece and said adaptation of the health monitor to measure a plurality of patient health parameters comprises a sensor selected from the group consisting of an oxygen saturation sensor, a body temperature sensor, a carbon dioxide level sensor, a pulse sensor, a blood glucose level sensor, an EKG sensor, an EEG sensor, a respiration sensor, a motion sensor, and any combination thereof.

5. The distributed cognitive system according to claim 4, wherein:
said earpiece comprises a light source and an optical sensor carried in suitable positions to receive a patient's earlobe there between when the earpiece is worn on a patient's ear, enabling parameter measurement through optical detection through the earlobe.

6. The distributed cognitive system according to claim 1, further comprising:
a remote computer at a care provider site, having capability for speech reception and speech output; and
means for remote bidirectional communication between said remote computer and said local computer;
wherein said health monitor and said sensor modules include microphone and speakers capable of exchanging spoken prompts over said means for local bidirectional wireless communication, whereby the remote computer is enabled to deliver spoken prompts through the health monitor and sensor modules.

7. The distributed cognitive system according to claim 1, wherein:
said adaptation of said sensor modules to measure ambient parameters comprises a sensor selected from the group consisting of an ambient temperature sensor, a photo sensor, an infrared sensor, a motion sensor, an occupancy sensor, a door position sensor, a medication delivery sensor, and any combination thereof.

8. The distributed cognitive system according to claim 1, further comprising:
a remote computer at a remote care provider site;
means for remote bidirectional communication between said remote computer and said local computer;
a point-of-care appliance providing interactive communications with a patient, including a display for presenting information, a calendar for scheduling activities, and a touch screen for interacting with a patient;
wherein said point-of-care appliance is in communication with said means for local bidirectional communication, enabling communications with the local computer, and is in communication with said means for remote bidirectional communication, enabling communications with the remote computer.

9. The distributed cognitive system according to claim 8, wherein said point-of-care appliance further comprises means for speech and voice recognition.

10. The distributed cognitive system according to claim 1, further comprising:
a point-of-care appliance providing capability for interactive communications with a patient, including
a display for presenting information;
a calendar for scheduling activities;
a touch screen for interacting with a patient;
a proximity detector triggering alerts;
timing means for scheduling measurements;
a speaker delivering audible alerts, voice prompts and messages;
a microphone for recording audible response;
processing means for interpreting measurements and delivering feedback responses; and
means for acting as a gateway to said health monitor, sensor modules, and local computer.

11. The distributed cognitive system according to claim 10, wherein said point-of-care appliance further comprises a sensor selected from the group consisting of an infrared temperature sensor, a motion sensor, an accelerometer sensor, magnetometer sensor, and any combination thereof.

12. A system adapted to deliver remote healthcare through medical monitoring and care intervention to a home-based patient, comprising:
a point-of-care appliance adapted for interactive communications with a patient, including:
a display for presenting information;
a calendar for scheduling activities; and
a touch screen for interacting with a patient;
a health monitor having a form factor suitable to accompany a patient and adapted to measure a plurality of patient health parameters;
a plurality of sensor modules suitable for deployment about the patient's surroundings and adapted to measure ambient parameters;
a local computer operating software establishing a cognitive system capable of receiving said measurements of patient health parameters and said measurements of ambient parameters, determining trends therefrom, reaching healthcare decisions, and issuing prompts to the patient; and
a wireless bidirectional gateway enabling communications between said health monitor, sensor modules, and local computer.
